SEQUENCE LISTING (2 sequences)

SEQ ID NO: 1 - DNA, 2546 bp, adult human aorta cDNA library
ggcacgagctgaagcggaagtggaggaaagatggaggaccatcagcacgtgcccatcgacatccagaccagcaagctgctcgattggctggtggacagaaggcactgcagcctgaaatggcagagtctggtgctgacgatccgcgagaagatcaatgctgccatccaggacatgccagagagcgaagagatcgcccagctgctgtctgggtcctacattcactactttcactgcctaagaatcctggaccttctcaaaggcacagaggcctccacgaagaatatttttggccgatactcttcacagcggatgaaggattggcaggagattatagctctgtatgagaaggacaacacctacttaggtaaagtggcccggcctgggagccctggtatccatggggaagcccactctcagagttctgagataccaggcttataggaggcacagtctgtgagtgggaagagactggagtgtagatgttgcccatttgtaggtggtaaaatcaattgtttttgatggaattgattttccctgagtggagtgctgggggaaggaggaggtccaggccggtagtggccattcgccgtgcctcagcgagcaggtgtgtgtgggtcctccaccactcacctcttggttagcgggagtgtgctgcccccacccccacccccgcacccccattctacacaaggcagaagaggcacgggttttcctgggagcgaatatcaagtgcctgagagcaactacaggactaactgtgtttgggttgggtgtagtataaataataataatggctaatatttcctgagcatctactaaatgcaaggaattgtgcttggtgtgtcatgtggattctctcttgcatcttcatgataaatgttattgtcgctgttttaccgatgagggttggattagaggggttaaacaacttgtcttaggctccacagctgggaacaagtggggctgggaagctgacttcgtgctcttcaccaccacaaaggatgtgtgtgcatcctggggcatgcctgcctcatgtgggggtgtcctgggctgaatttcctgggcacttctcagtggaactctctagcctcctggttcggaatgtcaactatgagatcccctcactgaagaagcagattgccaagtgccagcagctgcagcaagaatacagccgcaaggaggaggagtgccaggcaggggctgccgagatgcgggagcagttctaccactcctgcaagcagtatggcatcacgggcgaaaatgtccgaggagaactgctggccctggtgaaggacctgccgagtcagctggctgagattggggcagcggctcagcagtccctgggggaagccattgacgtgtaccaggcgtctgtggggtttgtgtgtgagagccccacagagcaggtgttgccaatgctgcggttcgtgcagaagcggggaaactcaacggtgtacgagtggaggacagggacagagccctctgtggtggaacgaccccacctcgaggagcttcctgagcaggtggcagaagatgcgattgactggggcgactttggggtagaggcagtgtctgaggggactgactctggcatctctgccgaggctgctggaatcgactggggcatcttcccggaatcagattcaaaggatcctggaggtgatgggatagactggggagacgatgctgttgctttgcagatcacagtgctggaagcaggaacccaggctccagaaggtgttgccaggggcccagatgccctgacactgcttgaatacactgagacccggaatcagttccttgatgagctcatggagcttgagatcttcttagcccagagagcagtggagttgagtgaggaggcagatgtcctgtctgtgagccagttccagctggctccagccatcctgcagggccagaccaaagagaagatggttaccatggtgtcagtgctggaggatctgattggcaagcttaccagtcttcagctgcaacacctgtttatgatcctggcctcaccaaggtatgtggaccgagtgactgaattcctccagcaaaagctgaagcagtcccagctgctggctttgaagaaagagctgatggtgcagaagcagcaggaggcacttgaggagcaggcggctctggagcctaagctggacctgctactggagaagaccaaggagctgcagaagctgattgaagctgacatctccaagaggtacagcgggcgccctgtgaacctgatgggaacctctctgtgacaccctccgtgttcttgcctgcccatcttctccgcttttgggatgaagatgatagccagggctgttgttttggggcctttcaaggcaaaagaccaggctgactggaagatggaaagccacaggaaggaagcggcacctgatggtgatcttggcactctccatgttctctacaagaagctgtggtgattggccctgtggtctaccaggcgaaaaccacagattctccttctagttagtatagcggacttaataaaagaggaaaaaacaaaaaaaaaaaaaaaaaaa

SEQ ID NO: 2 - protein, 419 aa, adult human aorta cDNA library
MCVHPGACLPHVGVSTAEFPGHFSVELSSLLVRNVNTEIPSLKKQIAKCQQLQQETSRKEEECQAGAAEMREQFTHSCKQTGITGENVRGELLALVKDLPSQLAEIGAAAQQSLGEAIDVTQASVGFVCESPTEQVLPMLRFVQKRGNSTVTETRTGTEPSVVERPHLEELPEQVAEDAIDTGDFGVEAVSEGTDSGISAEAAGIDTGIFPESDSKDPGGDGIDTGDDAVALQITVLEAGTQAPEGVARGPDALTLLETTETRNQFLDELMELEIFLAQRAVELSEEADVLSVSQFQLAPAILQGQTKEKMVTMVSVLEDLIGKLTSLQLQHLFMILASPRTVDRVTEFLQQKLKQSQLLALKKELMVQKQQEALEEQAALEPKLDLLLEKTKELQKLIEADISKRTSGRPVNLMGTSL

(19) United States
(12) Patent Application Publication
(10) Pub. No.: US 2004/0082031 A1
(43) Pub. Date: Apr. 29, 2004

(54) Inflammatory inhibition factor fwa116

(76) Inventors: Rutai Hui, Beijing (CH); Jingzhou Chen, Beijing (CH); Baohua Liu, Beijing (CH); Yuqing Liu, Beijing (CH); Yinhui Zhang, Beijing (CH)

Correspondence Address: Cherskov & Flaynik, The Civic Opera Building, Suite 1447, 20 North Wacker Drive, Chicago, IL, 60606, US

(21) Appl. No.: 10/469,625

(22) PCT Filed: Feb. 28, 2002
(86) PCT No.: PCT/CN02/00127

(30) Foreign Application Priority Data
Feb. 28, 2001 (CN) 01109262.9

(51) Int. Cl. C12P 21/02; C07H 21/04; C12N 9/99; C07K 16/40

(52) U.S. Cl. 435/69.1; 435/184; 435/320.1; 435/325; 530/388.26; 536/23.2

(57) ABSTRACT

The present invention relates to a polypeptide of the inflammatory inhibition factor Fwa116 and the polynucleotides encoding thereof. The present invention also relates to the method of producing polypeptide Fwa116 by recombinant technology and the antibody against it and an antagonist. The present invention also discloses pharmaceutical compositions containing the polypeptide Fwa116 and the use of the polypeptide in treating cardiovascular inflammatory arterosclerosis such as cerebral apoplexy, coronary heart disease, angina pectoris, myocardial infarction and controlling tumors. The present invention also provides the diagnosing and measuring method by detecting the mutation of nucleic acid sequence encoding Fwa116 or the content of the polypeptide Fwa116 and changes in levels of the auto-antibody of the gene product of Fwa116. The present inflammatory inhibition factor is human-derived.

### FIELD OF THE INVENTION

The present invention relates to the recently identified polynucleotide and its encoded polypeptide, and the method of producing said polynucleotide and polypeptide as well as the applications of said polynucleotide and polypeptide. The polypeptide of the invention has been identified as a cellular inflammatory inhibition factor, sometimes referred to as “Fwa116” hereinafter. The polynucleotide and polypeptide of the invention is human-derived.

### BACKGROUND

Cardiovascular and cerebrovascular diseases are among the most important diseases menacing human health. Statistics indicates that annual deaths of around 2.6 million Chinese are attributed to these diseases, and one compatriot is deprived of his life on average every 12 seconds. In China, the ratio of the deaths from cardiovascular and cerebrovascular diseases to the total deaths has risen from 12.1% in 1957 to 35.8% in 1990, an increase of 2.9 fold. According to an estimation by the World Bank, by 2020, the world-wide ratio of the deaths from cardiovascular and cerebrovascular diseases to the total deaths will increase to 36.3% from 28.9% in 1990, and 70% of cardiovascular and cerebrovascular diseases will occur in the developing countries. At the present time in China, there are more than 100 million hypertension patients with an annual increase of 3.5 million, 6 million disabled cerebral apoplexy patients with an annual increase of 1.5 million, 1 million coronary heart disease patients with an annual increase of 100 thousand, in addition to 3 million myocardosis patients. Therefore, preventing and treating cardiovascular and cerebrovascular diseases are of far reaching importance in relieving the individual economical burden and ensuring human health.

The treatments of cardiovascular and cerebrovascular diseases have passed through four main stages. In the 1920s, studies on circulation dynamics revealed that heart is a “pump”, by which the therapeutic basis of heart failure was established. In the 1960s-1970s, the incidence of heart disease in western countries declined by nearly 40% owing to the recognition and treatment of the risk factors of cardiovascular diseases. In 1970, studies on electrophysiology of myocardial cell provided new methods of resisting arrhythmia, electrophysiological detection and remedies. From the end of the 1980s to the beginning of the 1990s, knowledge about vascular biology promoted the occurrence of new methods of intervening heart therapy.

Heart failure, resisting arrhythmia, and intervening therapy play an active role in rescuing the lives of patients suffering from cardiovascular and cerebrovascular diseases, and improving their living quality. However, these methods only deal with symptoms of the diseases and do not touch the causes of the diseases in essence, as a result, they are less specific. Indeed, the purpose of prevention and healing cannot be achieved, although the lifetimes of patients are elongated. Therefore, researching on the level of molecular biology and finding out the pathogenic factors and mechanisms of cardiovascular and cerebrovascular diseases, are expected to bring about breakthrough progresses on the treatments of these diseases and achieve the purpose of restraining cardiovascular and cerebrovascular diseases in essence.

The cDNA library is one of the important research tools in the field of biotechnology. Generally, the mRNA isolated from the cell is used to synthesize the DNA copies (ie. cDNA, Complementary DNA) of said mRNA by reverse transcriptase. The resultant single strand cDNA molecules are then converted into double strand DNA molecules by DNA polymerase, which are subsequently inserted into a vector and transformed into the host cells, and then the clone is allowed to grow. In this way each clone only contains the information from the specific mRNA, and such a set of clones are referred to as the cDNA library.

Since there is no intron in cDNA, the corresponding expressed gene can be screened directly from the cDNA library. The cDNA library thus exhibits the advantages of simple operation and convenient use when compared to the gene library. The differences in the gene expressions in various human cells control the differences in the phenotypes of corresponding tissues and organs. Isolating and identifying specific expressed genes, especially isolating and identifying the disease-related genes from the cDNA library of human aorta, are effective means to investigate genetic cardiovascular diseases.

### SUMMARY OF THE INVENTION

According to one aspect of the invention, there is provided a novel mature polypeptide Fwa116 and its fragments, analogues and derivatives which have biological activity and are useful in diagnosis or treatment. The polypeptide of the invention is human-derived.

According to another aspect of the invention, there is provided the isolated nucleic acid molecules encoding the polypeptide of the invention, including mRNA, DNA, cDNA, genome DNA, and the fragments, analogues and derivatives of said nucleic acid molecules which have biological activity and are useful in diagnosis or treatment.

According to another aspect of the invention, there is provided a method for producing polypeptide Fwa116 by recombinant technology, comprising culture of recombinant prokaryotic and/or eukaryotic host cells containing the nucleic acid sequences encoding the polypeptide of the invention.

According to another aspect of the invention, there is provided a treatment method with polypeptide Fwa116 or polynucleotides encoding polypeptide Fwa116, for example, for treatment of cardiovascular inflammatory atherosclerosis.

According to another aspect of the invention, there is provided antibodies against said polypeptides.

According to another aspect of the invention, there is provided antagonists against said polypeptides, which can be used to inhibit the functions of said polypeptides.

According to another aspect of the invention, there is provided a method for diagnosing diseases and disease susceptibility relating to mutants in the nucleic acid sequences of the invention and abnormal expressions of the polypeptide of the invention.

According to another aspect of the invention, there is provided a method of applying the polypeptide of the invention and the polynucleotides encoding thereof to scientific research, DNA synthesis and artificial construction of DNA vectors in vitro.

According to the teachings herein, the aspects above and other related aspects are apparent to one skilled in the art.

The invention was carried out according to the protocols as follows: mRNA was isolated and then converted to cDNA by reverse transcription, and the cDNA library of the human aorta was constructed. Gene fragment of Fwa116 were obtained from the library and spliced by EST to produce the full-length cDNA sequence of Fwa116. Thereafter, the expression and distribution of the gene Fwa116 in various tissues and the activity and function of the polypeptide of Fwa116 were investigated.

### DESCRIPTION OF THE INVENTION IN DETAIL

According to one aspect of the invention, there is provided an isolated nucleic acid (polynucleotide) sequence encoding the mature polypeptide having the deduced amino acid sequence of SEQ ID NO:2. The polynucleotide according to the invention is found from the cDNA library of adult human aorta. It is situated on the No.17 chromosome of the human cell, comprising an open reading frame, encoding a polypeptide having 409 amino acid residues. Homology analysis demonstrates that polypeptide Fwa116 has 70% of homology with cdk5 activator binding protein C53.

The deduced polypeptide Fwa116 consists of 409 amino acids, the sequence of which is characterized by: (A) amino acids 13-18 (GVSWAE), amino acids 157-162 (GTEPSV), amino acids 193-198 (GTDSGI), amino acids 204-209 (GIDWGI) and amino acids 222-227 (GIDWGD) are N-myristoylation sites (5); (B) amino acids 15-18 (SWAE), amino acids 57-60 (SRKE), amino acids 113-116 (SLGE), amino acids 131-134 (SPTE), amino acids 150-153 (TVYE), amino acids 156-159 (TGTE), amino acids 161-164 (SVVE), amino acids 235-238 (TVLE), amino acids 255-258 (TLLE), amino acids 316-319 (SVLE) are casein kinase II sites (10); (C) amino acids 41-43 (SLK), amino acids 57-59 (SRK), amino acids 77-79 (SCK), amino acids 339-341 (SPR), amino acids 404-406 (SKR), amino acids 408-410 (SGR) are protein kinase C sites (6); (D) amino acids 148-151 (NSTV) is an N-glycosylation site (1); (E) amino acids 270-291 (LMELEIFLAQRAVELSEEADVL), amino acids 277-298 (LAQRAVELSEEADVLSVSQFQL) are leucine zipper motifs (2); and (F) amino acids 405-408 (KRYS) is a cAMP- and cGMP-dependent protein kinase site.

The polynucleotide according to the invention can be in RNA form or DNA form, wherein DNA includes cDNA, genomic DNA and synthetic DNA. DNA can be a double-strand or a single strand. If it is a single strand, it can be an encoding strand or non-encoding (antisense) strand. The mature polypeptide-encoding sequence can be identical to the encoding sequence (nucleotides 1002-2261) of SEQ ID NO:1 (2546 nucleotides in full length); alternatively, the encoding sequence can also be not identical to the encoding sequence of SED ID NO:1 due to the abundance or degeneracy.

The polynucleotide encoding the mature polypeptide of SEQ ID NO:2 can include: the mature polypeptide-encoding sequence; the mature polypeptide-encoding sequence and additional encoding sequences thereof, such as the polynucleotide encoding a leader sequence or a secretory sequence of the polypeptide; the encoding sequence (and optional additional sequence) and non-encoding sequence of the mature polypeptide, such as introns or the non-encoding sequence at 5′ and/or 3′ terminal of mature polypeptide-encoding sequences.

Accordingly, the term “polypeptide-encoding polynucleotide” includes the polynucleotide comprising the polypeptide-encoding sequence alone and the polynucleotide comprising the additional encoding and/or non-encoding sequences.

The invention also relates to the variants of the polynuclotide mentioned above, which encode the deduced polypeptide fragments having the amino acid sequence of SEQ ID NO:2, analogues and derivatives thereof. Said variants can be naturally occurring allele variants or non-naturally occurring variants of the polynucleotide. As known in the art, an allele variant is another form of a fragment of polynucleotide, and it can contain one or more substitution, deletion or addition of a nucleotide, but does not substantially alter the function of the encoded polypeptide thereof.

Accordingly, the invention not only includes the polynucleotide having the ability to encode the same amino acid sequence as that of the mature polypeptide of SEQ ID NO:2, but also includes the variants of the polynucleotide having the ability to encode the fragments of the mature polypeptide of SEQ ID NO:2, analogues and derivatives thereof. These variants include deletion variants, substitution variants, addition variants or insertion variants.

The invention also includes such a polynucleotide, wherein the mature polypeptide-encoding sequence can be fused in the same reading frame with the polynucleotides (e.g., the leader sequence-containing polynucleotide) which assist the host cell to express and secrete the polypeptides. The leader sequence can regulate the polypeptide to be transported from the cell as a secretory sequence. The polypeptide having a leader sequence is a preprotein, and can be converted into the mature polypeptide after the leader sequence thereof is cleaved by the host cell. The polynucleotide of the invention also encodes proprotein, which is a mature protein having a prosequence, with amino acid residues added at its 5′ terminal, and is inactive. After the prosequence is cleaved, the proprotein can yield an active mature protein.

Hence, the polynucleotide of the invention can encode a mature protein, the protein having prosequence, or the protein having both prosequence and leader sequence (presequence).

The polynucleotide of the invention also includes an encoding sequence fused with a marker sequence in the same reading frame, said marker sequence can also be used to purify the polypeptide of the invention. For example, when the host cell is a bacterium, the marker sequence can be six-histidine provided by vector pQE-9 and be used to purify the fusion product. Alternatively, when the host cell is a mammal cell (e.g., COS-7), the marker sequence can be a hemagglutinin (HA) corresponding to an epitope derived from influenza hemagglutination protein (Wilson, I. et al., Cell, 37:767 (1984)).

The term “gene” is referred to be the polypeptide-producing DNA fragment comprising the regions before and after the encoding region and insertion sequences (introns) between each encoding segment (exon).

The fragments of the full length gene of the invention can be used as hybridization probes of cDNA libraries to isolate the full length gene and other genes having high homology or similar biological activity with that of the full length gene. Preferably, said probes have at least 30 bases, and can have 50 or more bases. Said probes can also be used to identify the cDNA clones corresponding to the full length transcript and one or more genomic clones comprising the entire gene. The entire gene includes regulatory sequence, promoter sequence, exon and intron. For example, oligonucleotide probes can be synthesized according to the known DNA sequence, and the encoding portion of the gene is thus isolated. The marker oligonucleotide, being complementary with the gene sequence of the invention, can be used to screen its hybridized library member from the human cDNA, genomic DNA or mRNA libraries.

The invention also relates to the nucleic acid sequence hybridized with the polynucleotide of the invention, with the proviso that the two sequences have at least 85%, preferably at least 90%, more preferably 95% of homology with each other. In particular, the invention relates to the polynucleotide hybridized with the polynucleotide of the invention in the stringent condition. The term “stringent condition” as used herein is referred to be one in which only the sequences that have at least 95%, preferably at least 97% of homology with each other, can the hybridization occur. The polynucleotide hybridized with the sequences as indicated above can encode the polypeptide having the same biological function or activity as that of the mature polypeptide of the invention.

Furthermore, the polynucleotides having homology and ability to hybridize with the polynucleotide of the invention can have at least 20 bases, preferably at least 30 bases, more preferably at least 50 bases, and can remain active or unactive. Such polynucleotides can be used as the probes of SEQ ID NO:1 for recovery of polynucleotides, diagnostical probes or PCR primers.

Therefore, the invention relates to the pelynucleotides having at least 85%, preferably at least 90%, more preferably at least 95% of homology with that encoding the polypeptide of SEQ ID NO:2, the fragments thereof (said fragments have at least 30 bases, preferably at least 50 bases), and the polypeptides encoded by these polynucleotides.

According to another aspect of the invention, the invention relates to the deduced polypeptide having the amino acid sequence of SEQ ID NO:2, and the fragments, analogues and derivatives thereof.

The terms “fragment”, “derivative” and “analogue” are referred to be the polypeptides which retain substantially the biological function or activity when the terms relate to the polypeptide encoded by SEQ ID NO:1 or the polypeptide having the amino acid sequence of SEQ ID NO:2. Said analogues can include a proprotein which can be partially cleaved to convert into the mature polypeptide with activity.

The polypeptide of the invention can be a recombinant polypeptide, natural polypeptide or synthetic polypeptide, preferably a recombinant polypeptide.

Said polypeptide (SEQ ID NO:2) and the fragments, derivatives or analogues thereof can be: (i) a polypeptide wherein one or more amino acid residues are substituted by conservative or non-conservative amino acid residue (preferably conservative amino acid residue), and the substituted amino acid residue can or can not be encoded genetic code, (ii) a polypeptide wherein one or more amino acid residues contain substitution group, (iii) a polypeptide wherein the mature polypeptide is fused with another compound, for example, with the compound (such as polyethylene glycol) that increases the half life of the polypeptide, or (iv) a polypeptide wherein the mature polypeptide is fused with additional amino acid residues, for example, with a leader sequence or secretory sequence, and for example, with the sequence used to purify the mature polypeptide. Such fragments, derivatives and analogues can be considered to fall into the scope of those skilled in the art according to the teachings herein.

The polypeptide and the polynucleotide of the invention are preferably provided in an isolated form, and are preferably purified into homogeneous matters.

The term “isolated” is referred to be said matter separated from an original environment (for example, if the matter is naturally present, the original environment refers to natural environment). For example, a polynucleotide or a polypeptide naturally present in a living animal is not isolated, however, the same polynucleotide or polypeptide isolated from the partial or whole co-existing substances in the natural system is “isolated”. Such a polynucleotide can be a portion of the vector, and such a polynucleotide or polypeptide can be a portion of the composition, with the proviso that such a vector or composition is not a portion of the natural environment.

The polypeptide of the invention includes the polypeptide of SEQ ID NO:2 (particularly the mature polypeptide) and the polypeptides having at least 85%, preferably at least 90%, more preferably at least 95% of homology with the polypeptide of SEQ ID NO:2. The invention also includes the fragments of the polypeptides above which generally comprise at least 30, preferably at least 50 amino acids.

As known in the art, the “homology” between two polypeptides is determined by comparing the amino acid sequence and the substitution of conservative amino acids thereof of a polypeptide with that of another polypeptide.

The polypeptide fragments (or a portion of polypeptides) of the invention can be used to produce the full length polypeptide by polypeptide synthesis. The fragments can be used to produce the intermediates of the full length polypeptide. Also, the polypeptide fragments of the invention can be used to synthesize the full length polynucleotide of the invention.

According to another aspect of the invention, the invention relates to the vector containing the polynucleotide of the invention, the host cell genetically engineered with the vector of the invention, and the method for producing the polypeptide of the invention by recombinant technologies.

The host cell is obtained by genetically engineering (by transduction, transformation or transfection) method using the vector of the invention. Said vector can be a clone or an expression vector as a plasmid, viral particle, phage and the like. The engineered host cell can be cultured in an improved conventional nutrition medium to suitably activate the promoter, screen the tranformants or amplify gene Fwa116 of the invention. The culture conditions (e.g., temperature and pH), based on the different host cells, are apparent for those skilled in the art.

The polynucleotide of the invention can be used to produce the polypeptide by the recombinant technologies. The polynucleotide can be harbored in any one vector to suitably express the polypeptide. Such a vector includes chromosome-derived, non-chromosome-derived and synthetic DNA sequence, such as SV40 derivatives, bacterium plasmids, phage DNA, baculovirus, yeast plasmids, the vectors combining a plasmid DNA with a phage DNA, viral DNAs (e.g., vaccinia, adenovirus, fowl poxvirus and pseudo-rabies virus). Furthermore, other vectors can also be used if they can be replicated and live in the host.

Various procedures can be used to incorporate the suitable DNA sequence into the vectors. Generally, the known procedures in the art can be used to incorporate the DNA sequence into the suitable restriction endonuclease sites.

The DNA sequence of the expression vector can be ligated with a suitable expression-controlled sequence (promoter) so as to guide the synthesis of mRNA. The examples of such a promoter are LTR or SV 40 promoters, lac or trp of E. coli, λP_{L} promoter of phage, and other known promoters to regulate the gene expression in prokaryotic or eukaryotic cells or the viruses therein. The expression vector also includes the ribosome-binding site and transcription terminator to guide the initiation of translation. The vector also includes the suitable sequence for amplification and expression.

Further, the preferable expression vector includes one or more selective marker genes to provide the phenotype characteristic for the selection of the host cell after it is transformed, for example, di-hydrogen folic acid reductase or neomycin resistance for the culture of eukaryotic cell and tetracycline and ampicillin resistance for the culture of E. coli.

The vector comprising a suitable DNA sequence, suitable promoter or regulation sequence as mentioned above can be used to transform a suitable host so as to express proteins.

The representative examples of suitable hosts are bacterium cells, such as E. coli, streptomyce, Salmonella typhimurium□fungus cells, such as yeast; insect cells, such as Drosorphila S2 and Spodoptera Sf9; animal cells, such as CHO, COS or Bowes melanoma; adenovirus; plant cells and the like. The selection of a suitable host is considered to fall into the scope of those skilled in the art by the teachings of this invention.

More specifically, the invention also includes the recombinant construct having one or more sequences as mentioned above. The construct includes the vector inserted forward or backward with the nucleic acid sequence of the invention, e.g. a plasmid or viral vector. In a more desirable embodiment, the construct also includes the regulation sequence operatively linked with the above sequence(s), e.g. a promoter. Many suitable vectors and promoters are known by those skilled in the art, and can be commercially obtained. For example, bacterium vectors: pQE70, pQE60, pQE-9 (Qiagen), pBS, pD10, phagescript, psiX174, pbluescript SK, pbsks, pNH8A, pNH16a, pNH18A, pNH46A (Stratagene), ptrc99a, pkk223-3, pkk233-3, pDR540, pRITS (Pharmacia); eukaryotic vectors: pWLNEO, pSV2CAT, p0G44, pXT1, pSG (Stratagene), pSVK3, pBPV, pMS, pSVL (pharmacia). Further, other plasmids or vectors can also be used if they can be replicated and live in the host.

The promoter regions can be selected from the genes with the vectors carrying chloramphenicol acetyltransferase (CAT) or other selective markers. Two suitable vectors are PKK232-8 and PCM7. The special bacterium promoters include lacd, lacZ, T3, T7, gpt, λP_{R}, P_{L} and trp. The eukaryotic promoters include CMV immediate early promoter, SV thymidine kinase promoter, early and late SV40 promoter, LTRs promoter derived from retrovirus and mouse metallothionein-I promoter. The selection of suitable vectors and promoters is considered to fall into the scope of those skilled in the art.

In another embodiment, the invention relates to the host cell carrying the above construct. The host cell can be higher eukaryotic cells (e.g. mammal cell) or lower eukaryotic cells (e.g. yeast), or prokaryotic cells (e.g. bacterium cell). The incorporation of the construct into the vector can be performed by phosphate calcium transfection, DEAE-glucan meditated transfection or electroporation method (Davis, L., Dibner, M., Battery, I., the Basic Procedures of Molecular Biology, (1986)).

The construct of the host cell can produce a product encoded by recombinant sequence in a conventional manner. Further, the polypeptide of the invention can be synthesized by the common polypeptide synthesizer.

Under the control of the suitable promoter, the mature protein can be expressed in a mammal cell, yeast cell, bacterium cell or other cells. The protein of interest can also be produced by a cell-free translation system with RNA derived from the DNA construct of the invention. The suitable clones and expression vectors for the prokaryotic host and eukaryotic host are described in Sambrook et al., the Laboratory Manual of Molecular Cloning, 2^{nd} edition, 1989, Cold Spring Harbor Laboratory, New York.

Higher eukaryotic cells can improve the transcription of the DNA encoding the polypeptide of the invention by introducing an enhancer sequence into the vector. The enhancer is a cis-acting element of DNA, usually approximately 10-300 bp. It acts on the promoter to the enhance transcription thereof. The examples of the enhancer are SV40 enhancer situated in 100-270 bp upstream of the replication origin, polymorphous tumor enhancer and adenovirus enhancer.

In general, the recombinant expression vector includes a replication origin and selective marker gene (e.g., ampicillin resistance gene of E. coli and TRP1 gene of Saccharomyces cerevisiae), and the promoter obtained from a highly expressed gene and capable of guiding downstream structure gene transcription. Such a promoter can be obtained from the operons encoding the saccharolytic enzyme (e.g., 3-phosphoglycerate kinase (PGK)), α-factor, acid phosphatase or heat shock protein. A heterologous sequence is assembled with a translation-initiating sequence and a termination sequence in an exact manner. Preferably, it is assembled with the leader sequence having the ability to guide the protein to secrete into cytoplasma or extracelluar medium. The heterologous sequence can encode the fusion protein comprising N-terminal cognitive peptide, which is desirably characterized by, for example, the stably expressed recombinant product or simplified purification step.

The expression vector for bacterium can be constructed by inserting the structure genes encoding protein of interest, a suitable translation initiation and termination signal, and a functional promoter. Said vector comprises one or more selective markers and a replication origin to maintain the vector and to amplify the vector in the host, if necessary. A suitably transformed prokaryotic host includes E. coli, Bacillus subtilis, Salmonella typhimurium, and various species of pseudomonas, streptomyces and staphylococcus.

As a representative but not limited example, the expression vector for the bacterium can comprise the selective markers and replication origins from commercial vectors containing known genetic elements of cloning vector pBR322 (ATCC37017). Such commercial vectors include pKK223-3 (Pharmacia Fine Chemical Co., Uppsala, Sweden) and GEMI (Promega Biotec, Madison, Wis., USA). The “backbone” portion of pBR322 can be combined with a suitable promoter and the structure sequence to be expressed.

When the suitable host strain is transformed and cultured to a proper cell density, the selective promoter is induced in an exact manner (e.g., temperature change or chemical induction), and the cell continues to be cultured for a period. Usually, the cells are collected by a centrifuge method and disrupted by a physical or chemical method, and thus the resultant crude products remain for further treatment. Any of the conventional methods can be used to disrupt microbial cells, said methods include a freeze-thaw method, ultrasonic treatment, mechanical disruption or a cytolysis agent. These methods are well known for those skilled in the art.

Various mammal cell culture systems can also be used to express recombinant proteins. The examples of the mammal expression systems are monkey's renal fibroblast COS-7 cell line described in Gluzman (Cell, 23:175 (1981)) and other cell lines having the ability to express compatible vectors, such as C127, 3T3, CHO, HeLa and BHK cell lines. The mammal expression vectors include the replication origin, suitable promoter and enhancer, as well as any necessary ribosome-binding site, polyadenylated site, splicing donor and recipient site, transcription termination sequence and 5′ flanking non-transcription sequence. The DNA sequence obtained from SV40 viral genome, such as SV40 replication origin, early promoter, enhancer, splicing site and polyadenylated site, can be used to provide the necessary non-transcriptional genetical elements.

Various methods can be applied to recover and purify the polypeptide of the invention from the culture of recombinant cells. Said methods include ammonium sulfate or ethanol precipitation, acid extraction, anion- or cation-exchange chromatography, phosphocellulose chromatography, hydrophobic chromatography, affinity chromatography, hydroxyapatite chromatography, plant agglutinin chromatography and high performance liquid chromatography (HPLC).

The polypeptide of the invention can be naturally purified or chemically synthesized or prepared from a prokaryotic or eukaryotic host (e.g., bacterium, yeast, higher plant, cultured insect and mammal cell) by the recombinant technologies. According to the host used in the recombinant method, the polypeptide of the invention can be glycosylated or non-glycosylated. The polypeptide of the invention can also comprise an initial methionine residue.

The polynucleotide and the polypeptide of the invention can be used as the research reagents and materials for the treatment and diagnosis of human diseases. Fwa116 plays a role in inhibiting inflammation and can be used to treat cardiovascular inflammatory arterosclerosis such as cerebral apoplexy, coronary heart disease, angina pectoris, myocardial infarction and to control tumors.

According to another aspect of the invention, there are provided methods for identifying the polypeptide agonist or antagonist of the invention. According to one of the methods, if a certain compound is present, the Fwa116 acceptor-expressing mammal cell or membrane formulation is incubated with Fwa116, then the ability to produce a second messenger is determined after the compound promotes or blocks polypeptide Fwa116 to react with the acceptor. The second messengers include, but are not limited to, protein tyrosine kinase system (PTK), cAMP, cGMPase, ion channel or phosphoinositide hydrolysis. Another method for identifying polypeptide antagonist is the competitive inhibition method. According to the method, the potential antagonist is determined by detecting the change in the number of polypeptide Fwa116 molecules bound with the acceptor when a certain compound is present, while the number of polypeptide Fwa116 molecules is set to a control amount when the compound is absent.

Potential antagonists include antibodies, otherwise they include the oligopeptides bound with the polypeptide of the invention in some cases. These antibodies or oligopeptides can bind with said polypeptide and eliminate the function thereof in an effective way.

Another potential antagonist compound is an antisense construct prepared by the antisense technology. Due to formation of antisense DNA or RNA by three strands of helix to control the expression of the gene, all the methods above are based on the binding of the polynucleotide with DNA or RNA. For example, the antisense RNA of about 10-40 bases in length can be designed according to the 5′ terminal of the nucleotide sequence encoding the mature polypeptide of the invention. Again, a complementary DNA with transcription-involving gene region (three helix, see, Lee et al., Nucleic Research, 6:3073 (1979); Cooney et al., Science, 241:456 (1988); and Dervan et al., Science, 251:1360 (1991)) is designed to prevent the transcription and the production of the polypeptide of the invention. Antisense RNA hybridizes with mRNA in vivo, and thus blocks the mRNA molecule translation into the polypeptide of the invention (antisense-Okano, J. Neurochemistry, 56:560(1991); deoxy-oligonucleotide as a gene expression antisense inhibitor (CRC press, Boca Raton, Fla.(1988)). The oligonucleotide above can be delivered to cells to express antisense RNA and DNA in vivo to inhibit the production of the polypeptide of the invention.

The antagonists also include some small molecules bound with the polypeptide of the invention to prevent the reaction between the polypeptide and acceptor thereof to block its normal biological activities. Small molecules include, but are not limited to, small peptide or peptideoid molecules.

The polypeptide of the invention, agonist and antagonist thereof can form a pharmaceutical composition in combination with suitable carriers. Such a composition comprises a therapeutically effective amount of the polypeptide and pharmaceutically acceptable carriers or recipients. The carriers include, but are not limited to, saline, buffer, glucose, water, glycerol, ethanol and the combination thereof. Their formulation should be in conformity with the administration route.

The invention also provides a pharmaceutical package or kit comprising one or more containers therein, in which one or more components of the pharmaceutical composition of the invention is contained. Also provided is the information on the related manufacture, application and sale of medicine or a biological preparation approved by the government drug administration authorities.

The pharmaceutical composition can be administered in a conventional manner, such as orally, locally, intravenously, intraperitoneally, intramuscularly, subcutaneously, intranasally or dermally. The pharmaceutical composition is administered in an effective amount to cure and/or prevent a specific disease. It is often administered in an amount of at least about 10 μg/kg body weight. In most cases, it is administered in less than about 8 mg/kg of body weight per day. In most cases, the dosage of administration is about 10μg/kg—1 mg/kg of body weight per day when the administration route and conditions are taken into the consideration.

According to another aspect of the invention, the polypeptide of the invention and agonists and antagonist thereof can be used in an expression manner in vivo, also often called “gene therapy”.

Hence, the patient's cells can be genetically engineered in vitro with the nucleic acid (DNA or RNA) encoding the polypeptide of the invention, then the engineered cells are provided to the patients in need thereof. The method above is well known in the art. For example, the cells can be genetically engineered with the retrovirus comprising the RNA encoding the polypeptide of the invention.

Similarly, the cells can be genetically engineered in vivo by the known method in the art to express the polypeptide in vivo. For example, the packaging cells can be transduced with the retrovirus comprising the RNA encoding the polypeptide of the invention to enable them to produce infectious viral particles comprising the gene of interest. The resultant cells can be used for the patients such that the cells can be engineered in vivo and express said polypeptide. According to the teachings of the invention, it is apparent for those skilled in the art to administer the polypeptide of the invention by the methods above or other ways.

The retroviruses that can acquire retrovirus plasmid vectors include, but are not limited to, Moloney murine leukemia virus, spleen necrosis virus, retrovirus, such as Rous sarcoma virus, Harvey sarcoma virus, Avian leukosis virus, Gibbon leukosis virus, Human immunodeficiency virus, Adenovirus, Myelcytomatosis virus and Mammary tumor virus.

Said vectors include one or more promoters. The suitable promoters used include, but are not limited to, retrovirus LTR, SV40 promoter, Human cytomegalovirus (CMV) promoter (Miller et al., biotechnology, Vol.7, No.9, 980-990 (1989), and other promoters (e.g., eukaryotic cell promoters, including, but not limited to, histone, pol III and β-actin promoter). Other virus promoters employed include, but are not limited to, Adenovirus promoter, thymidine kinase (TK) promoter and B19 picornavirus promoter. It is obvious for those skilled in the art to select suitable promoters for the vectors by the teachings of the invention.

The nucleic acid sequences encoding polypeptide of the invention should be controlled by suitable promoters. The suitable promoters employed include, but are not limited to, Adenovirus promoter (e.g., the main late promoter of adenovirus); or heterologous promoters (e.g., cytomegalovirus (CMV) promoter); respiratory syncytial virus (RSV) promoter; inducible promoters (e.g., MMT promoter, metallothionein promoter); heat shock promoter; albumin promoter; ApoA□promoter; human bead protein promoter; virus thymidine kinase promoter (e.g., herpes simplex virus thymidine kinase promoter); retrovirus LTRs (including modified retrovirus LTRs described above);β-actin promoter and human hormone promoter. The promoter can also be the natural promoter of the gene encoding said polypeptide.

The retrovirus plasmid vectors can be transduced into the packaging cells to generate producing cells. Such packaging cells include, but are not limited to, PE501, PA317,ψ-2,ψ-AM, PA12, T19-14X, VT-19-17-H₂,ψCRE,ψCRIP, GP⁺E-86, GP+envAm12 and DNA cell line (Miller, Human Gene Therapy, Vol.1, pages 5-14(1990), all the contents are incorporated by reference in their entirety). The vectors can be transduced into the packaging cells by any known methods in the art. These methods include, but are not limited to, electroporation, liposome and CaPO₄ precipitation. In addition, the retrovirus plasmid vectors can be embedded into the liposome, or conjugated to the lipid, then introduced into the hosts.

The resultant cell lines generate infectious retrovirus vector particles comprising nucleic acid sequence encoding said polypeptide. These retrovirus vectors can be transduced into eukaryotic cells in vivo or in vitro. The transduced eukaryotic cells will express the nucleic acid sequence encoding said polypeptide. The eukaryotic cells that can be transduced include, but are not limited to, embryonic stem cell, embryonic cancer cell, hemopoietic stem cell and liver cell, fibroblast, myoblast, keratirocyte, endothelial cell and bronchial epithelial cell.

According to another aspect of the invention, the invention relates to the use of the Fwa116 gene in diagnosis or detection. The related diseases and susceptibility to the diseases can be diagnosed by detecting the mutation of the Fwa116 nucleic acid sequence.

Various techniques can be used to detect the individual carrying the mutation of the Fwa116 gene. The genome DNA of patient cells such as these derived from blood, urine, saliva, biopsy and autopsy material can be directly detected, or can be amplified by PCR before the analysis (Saiki et al., Nature, 324:163-166 (1986)). RNA or DNA can also be used for the same purpose. For example, the PCR primers complementary with the polynucleotide of the present invention can be used for identification and analysis of the mutation. For example, deletion and insertion can be detected based on the size change of amplified products by comparing them with the normal genotype. Further, the point mutation can be identified by hybridization of radioactive labelled RNA or antisense DNA and the amplified nucleic acid sequence. The perfectly matched sequence and the mis-matched double strands can be distinguished from each other via RNase digestion or the difference of the strand-melting temperature.

The sequence difference between the control gene and the mutant gene can be directly indicated by DNA sequencing. In addition, the cloned DNA fragments can also be used as probes to detect specific DNA regions. The sensitivity of the method was highly improved in combination with PCR. For example, the sequencing primers can be used together with double PCR products or single strand template molecules generated by the improved PCR method. The nucleic acid sequence can be determined by a radioactive label or a fluorescence label in the conventional auto sequencing.

The genetic assay based on the DNA sequence difference can be achieved by detecting the variation of electrophoresis mobility of DNA fragments in the gel with or without denaturants. The short sequence deletion and insertion can be shown by gel electrophoresis of high resolution. DNA fragments of different sequences can be distinguished in denaturing formamide gradient gel electrophoresis. The different DNA fragments will retard in the different places of the gel according to the specific melting point and partially strand-melting temperature (see Myers et al., Science, 230:1242(1985)).

The nuclease protection analysis method through RNase and S1 protection or the chemical cleavage method can also detect the variation of the sequence on the special location (e.g., Cotton et al., PNAS, USA, 85:4397-4401 (1985)).

Accordingly, the DNA sequence difference can be detected by hybridization, ribonuclease protection, chemical cleavage, direct DNA sequencing or restriction enzyme (e.g., restrictive fragment length polymorphism (RFLP)) and Southern blotting of genome DNA.

Besides, more conventional gel electrophoresis and DNA sequencing, the mutation can also be detected by in situ analysis.

According to another aspect of the invention, the present invention relates to a diagnosis and analysis method by detecting the variation of the content of polypeptide Fwa116 in different tissues. Based on comparison with normal tissues, the overexpression of said polypeptide in some tissue can detect the presence of the disease or the disease susceptibility. The assay methods for the detection of the content of the polypeptide of the invention from the samples of a host is well known by those skilled in the art. The methods include radioimmunoassay, competitive binding assay, Western blotting, Enzyme-linked immunoabsorbent assay (ELISA) and “sandwich” assay, preferable Enzyme-linked immunoabsorbent assay. Enzyme-linked immunoabsorbent assay comprises firstly preparation of the specific antibody of the polypeptide of the invention, preferably the monoclonal antibody, and then preparation of the reporter antibody of said monoclonal antibody. The reporter antibody is combined with a detectable agent, such as radioactive agent, fluorescent agent or horseradish peroxidase. The sample was extracted from the host, and then incubated in the solid support (e.g., polystyrene plate) binding with the protein of the sample. Any free protein-binding sites were covered in the plate by incubating with the non-specific proteins (e.g., bovine serum albumin). Next, during the combination of the monoclonal antibody with any polypeptide of the invention binding to polystyrene plate, monoclonal antibody was incubated in plate. All the unbinding monoclonal antibodies were washed with the buffer. At this time, the receptor antibodies linked with horseradish peroxidase were put into the plate, which resulted in the combination of the receptor antibody and any monoclonal antibody binding to the polypeptide of the invention. The unbinding monoclonal antibodies were washed again. Then the substrate of peroxidase was added into the plate. Compared with the standard curve, the quantity of color generated in the given time is the quantity of the presence of the protein of the sample from the patients in the given volume.

Competitive assay can also be used to detect the content of the polypeptide. Said assay comprises binding of the specific antibody of polypeptide Fwa116 to the solid support, then labeling (e.g., radioactive labeling) the polypeptide of the invention, then enabling the sample to pass from the host through the solid support, and then determining the quantity of the sample competitively binding with the antibody by detecting the labeling amount to determine the content of the polypeptide of the invention from the sample.

The polynucleotide sequence of the invention is very valuable to discriminate chromosomes. Said sequence is specifically targeted to the specific location of the human chromosome and hybridized with the human chromosome. At present, only several chromosome marker agents based on actual sequence data can be used to label the location of the chromosome. Chromosome mapping based on the DNA of the invention is the primary step to associate these sequences with the disease-related genes.

In brief, the sequences can be located on the chromosome by preparing the PCR primers (preferable 15-25 bp) from cDNA. The primers can be quickly selected by analysis of the 3′ non-translated region in computer. The primers should not span the first exon of genome DNA, otherwise they will complicate the amplification. Then, the primers were used in PCR to screen the somatic cell hybrid containing a single human chromosome. Only the hybrid containing the genes corresponding to said primers generates the amplified fragments.

PCR mapping of somatic cell hybrid is a quick method to locate the specific DNA on the specific chromosome. According to the invention, the same oligonucleotide primers and a set of fragments from the specific chromosome or the large genome clone can be used for sub-location according to the similar method. The other chromosome mapping methods include in situ hybridization, prescreening by the labeled and flow sorting chromosome, and prescreening by hybridization, so as to construct the chromosome-specific cDNA library.

Fluorescence in situ hybridization (FISH) of cDNA clone and metaphase chromosome smear can achieve more accurate chromosome locating. The technique may employ 50 or 60 bp cDNA. See the review of Verma et al., Human Chromosome: Basic Technique Manual, Pergamon press, New York (1988).

The physical location of said gene in the chromosome can be associated with the data of the genetic map, once the gene was located accurately on the chromosome. This data may be found in for example, V. Mckusick, Human Mendel-like Heredity (acquired from Welch medical library of Johns Hopkins University via internet). Then the relation between the gene and the disease that was located on the same region of the chromosome can be determined by linkage analysis (co-heredity of physically contiguous genes).

The cDNA or genome sequence difference between the patient individual and the normal individual was then determined. If the mutation can be observed in the whole or part of the patients, while the mutation can not be found in the normal individuals, said mutation may be the cause of the disease.

Said polypeptide, and fragments, derivatives thereof and the like, or the cell expressing these substances can be used as immunogen to induce antibody generation. The antibody can be a polyclonal or a monoclonal antibody. The invention also includes the chimeric, single-strand and humanization antibody as well as Fab fragments or the products of Fab expression library. Many methods known in the art can be used to generate these antibodies and fragments.

The corresponding antibody can be obtained by the direct injection or the administration of the polypeptide of the invention to the animals (preferable non-human). The resultant antibody can combine with said polypeptide whereby, the sequence encoding the fragments of the polypeptide may even generate antibodies that can bind with the whole natural polypeptide. Further, the polypeptide can be used to isolate the polypeptide from said polypeptide-expressing tissue.

Any techniques for the production of the antibodies by continuous culture of the cell line can be employed in order to prepare the monoclonal antibody. For example, hybridoma technique (Kohler and Milstein, 1975, Nature, 256:495-497), three-body hybridoma technique, human B-cell hybridoma technique (Kozbor et al., Today Immunology, 4:72) and EBV-hybridoma technique (Cole et al., 1985, Monoclonal Antibody and Cancer Therapy, Alan R. Liss, Inc., pp,77-96).

The technique for the production of single-strand antibody (U.S. Pat. No. 4,946,778) can be improved to generate the single-strand antibody against the polypeptide of the invention (immunogenicity). The humanization antibody against the polypeptide of the invention (immunogenicity) can be expressed in transgenic mice.

The invention will be explained in more detail with reference to the following figures and examples to clearly understand the spirit of the invention. The figures and examples are intended to illustrate but not limit the invention in any way.

### BRIEF DESCRIPTION OF THE FIGURES

FIG. 1 shows the distribution of Fwa116 in the normal tissue, wherein A is Fwa116; and B isβ-actin (control).

FIG. 2 shows the distribution of Fwa116 in the various tumor cell strains, wherein A is Fwa116; and B isβ-actin.

FIG. 3 shows the effects of concentration of oxidized low density lipoprotein (ox-LDL) and genistein on endothelial cell expressing Fwa116, wherein A is the reaction result; and B is the loading amount.

FIG. 4 shows the expression of Fwa116 in the normal, sub-acute heart failure and chronic heart failure animal aorta, wherein A is normal animal aorta; B is sub-acute heart failure animal aorta; and C is chronic heart failure animal aorta.

FIG. 5 shows the expression of Fwa116 in the normal, sub-acute heart failure and chronic heart failure animal cardiovascular system, wherein A is normal animal cardiovascular system; B is sub-acute heart failure animal cardiovascular system; and C is chronic heart failure animal cardiovascular system.

FIG. 6 shows the expression of Fwa116 in the normal myocardial tissue and acute myocardial infarction ventricular aneurysm tissue, wherein A is normal myocardial tissue; and B is acute myocardial infarction ventricular aneurysm tissue.

FIG. 7 shows the overexpression of Fwa116 gene induces the propagation of ECV 304 cell.

### EXAMPLES

### Example 1

### Construction of cDNA Library from Adult Human Aorta

### 1.1 Extraction of RNA

RNA gents® Total RNA Isolation System kit was purchased from Promega Co. of USA (Cat No.Z5110). Procedures were provided as follows: 0.3 g of tissue of adult human aorta stored in liquid nitrogen was weighed, 10 mL of denaturing solution (4M guanidine isosulfocyanate, 25 mM tri-sodium citrate), and 1 mL of 2M sodium acetate (pH4.0) were added and homogenized. An equal volume of water-saturated phenol and 0.2 volume of chloroform were added, vertexed vigorously for 15 seconds and then placed on ice for 15 minutes. The mixture was separated by centrifugation at 10,000 rpm for 20 minutes at 4□. To the supernatant an equal volume of isopropanol was added and placed at −20□ for 2 hours and centrifuged again. The precipitate was resuspended in 5 mL of denaturing solution and the previous steps were repeated. The precipitate was washed with 1 mL of ice pre-cooled 75% ethanol, and trace ethanol was evaporated for 5-10 minutes at room temperature. RNA was dissolved with diethyl pyrocarbonate (referred to as DEPC thereinafter)-treated deionized water.

### 1.2 Isolation of mRNA

At the 3′ terminal of mature mRNAs, there was a poly (A) sequence consisting of 20-250 adenosines, by which mRNAs can be separated from other RNAs through affinity chromatography. Oligo (dT)-cellulose used in the process consisted of a polymer chain with 12-18 nucleotides (T). In high salt conditions, mRNAs with an oligo (A) tail could bind to oligo (dT) and retain in the column, while rRNA and tRNA without the oligo (A) tail were washed out, and then the mRNAs binding on the column could be eluted by low salt solution.

Quick prep® micro mRNA purification kit was purchased from Pharmacia Co. (Sweden). 1 mL of oligo(dT)-cellulose suspension was added to a 1.5 mL RNase-free centrifuge tube 1#; and 1 mL of Total RNA solution diluted by loading buffer was added to another 1.5 mL centrifuge tube 2#; tubes 1# and 2# were centrifuged at 12,000 rpm for 1 minute at room temperature; the supernatant in tube 1# was removed by aspiration, and the supernatant in tube 2# was transferred to tube 1#, tube 1# was mixed by gently shaking for 5-10 minutes; centrifuged at 12,000 rpm for 10 seconds at room temperature; washed with 1 mL of high salt buffer (10 mM Tris-HCl pH7.5, 1 mM EDTA, 0.5M NaCl) for five times and centrifuged again; washed with 1 mL of low salt buffer (10 mM Tris-HCl pH7.5, 1 mM EDTA, 0.1 M NaCl) for five times and centrifuged; the precipitate of oligo(dT) was suspended in 0.3 mL of low salt buffer and transferred to a mini centrifuge column, then centrifuged at 12,000 rpm for 5 seconds; washed with 0.5 mL of low salt buffer for three times and centrifuged again; mRNAs were eluted and collected with 2×0.2 mL of elution buffer; the ratio of OD260/280 was calculated according to the optical density determined by spectrophotometer; 7.5 μL of glycogen, 30 μL of 2.5M potassium acetate(pH5.0) and 750 μL water-free ethanol were added to 300 μL of the resultant mRNA, and stored at −70□ until use; prior to use, mRNA was centrifuged for 5 minutes and washed with 75% ethanol, and then dissolved into DEPC-treated water.

### 1.3 Synthesis of cDNA

The procedures of synthesis were performed with reference to the manufacturer's instructions of ZAP Express™ cDNA Synthesis Kit and ZAP Express™ cDNA Gigapack® II Gold Cloning Kit (Stregene Co., USA, Catalog No.200403 and 200404).

5 μL of 10×buffer for synthesis of first-strand cDNA, 3 μL methylated dNTP mixture, 2 μL of XhoI linker oligo (dT) 18 primer (1.4 μg/μL), 32.5 μL of DEPC-treated deionized water, 1 μL of RNase inhibitor (40 u/μL), and 5 μg of mRNA were sequentially added to a 0.5-mL centrifuge tube and placed for. 10 minutes at room temperature. Then 1.5 μL of Moloney murine leukemia virus (MMLV) reverse transcriptase (50 u/μL) was added to a total volume of 50 μL. 5 μL of the reaction was transferred to another 0.5-mL centrifuge tube and 0.5 μL of [α-³²P]dATP (800 ci/mmol) was added to identify the synthesis quality ofthe first strand. All the reactions described above were performed at 37□.

The second-strand of cDNA was synthesized using the first-strand cDNA as a template by DNA polymerase I based on the resultant DNA/RNA hybrid molecules. 45 μL of first-strand cDNA, 20 μL of 10×buffer for synthesis of second-strand cDNA, 6 μL of dNTP mixture, 114 μL of deionized water, 2 μL of [α-³²P]dATP (800 ci/mmol), 2 μL of RNase H(1.5 u/μL), and 1 μL of DNA polymerase I (9.0 u/μL) were sequentially added, with a total volume of 200 μL, and mixed evenly. The reaction was incubated for 2.5 hours at 16□. At the end of incubation, the reaction solution was extracted with phenol-chloroform (1:1) and precipitated with ethanol.

### 1.4 Ligation of the cDNA with Vector

The precipitate was dissolved in 9 μL of EcoRI linkers (their sequences were 5′-AATTCGGCACGAG-3′ and 3′-GCCGTGCTC-5′, respectively) from the kits. 1 μL of the mixture was taken out and analyzed by electrophoresis to identify the synthesis quality of the first-strand and second-strand, and 1 μL of 10×ligation buffer, 1 μL of 10 mmol/L γ-ATP, and 1 μL of T4 DNA ligase (4 ul/μL) were sequentially added to the remaining 8 μL of second-strand cDNA. After incubating for 16 hours at 8□, the mixture was incubated for another 30 minutes at 70□. After being digested by restriction enzyme XhoI for 1.5 hours, the reaction mixture was separated by Sepharose CL-2B chromatograph to remove the polynucleotides less than 400 bases to increase the proportion of full-length cDNA in the cDNA library.

### 1.5. Cloning of cDNA into ZAP Phage Vector

The polycloning site located in the ZAP phage vector (Stratagene Co., USA) allows the insertion of nucleic acid fragments of 10 Kb in length. Having been introduced into the host, the portion of plasmid can be cut off the vector to create the plasmid vector Bluescript. The T7 and T3 bacteriophage promoters located at both flanks of said polycloning site can be used for analysis of sequences and synthesis of probes. The cDNA fragments inserted in the vector can express fusion proteins with antigenicity and biological activity. The procedures are provided in detail as follows: 100 ng of cDNA, 0.5 μL of 10×ligation buffer, 0.5 μL of 10 mmol/L γ-ATP (pH7.5), 1 μL of ZAP phage vector (1 μg/μL) and 0.5 μL of T4 DNA ligase (4 u/μL) were added to a centrifuge tube, and deionized water was added to a total volume of 5 μL. Ligation reaction was performed for 16 hours at 12□.

It is necessary to package the ligated product with packaging proteins to generate recombination phage with transfection activity. The packaging proteins stored at −70□ were quickly taken out. Having been thawed, 25 μL of packaging proteins was added to 1 μL of ligation reaction and mixed evenly, reacting for 2 hours at 22□, and 500 μL of SM buffer (0.1M NaCl, 0.08M MgSO₄.7H₂O, 0.05M Tris-HCl, 0.01% gelatin) and 20 μL of chloroform were added. The resultant mixture was the original cDNA library.

### 1.6 Calculation of the Titre of Positive Clones

5 μL of original cDNA library was diluted with 45 μL of SM buffer. 10 μL of the diluted solution was added to 200 μL of competent XL1-Blue MRF′ host cells (OD₆₀₀=0.5). After incubating in a water bath for 20-30 minutes at 37□, 3 mL of upper layer agarose solution was added and mixed evenly, then plated on the plates containing NZY medium (0.09M NaCl, 0.08M MgSO₄.7H₂O, 0.5% yeast extract, 1% NZY). The plates were placed upside down and cultured overnight at 37□, then the number of clones on the plates was calculated.

The titre of cDNA library was expressed as plaque forming units (pfu/mL): pfu/mL=(number of plaques×dilution factor)×1000/volume of dilute solution used (μL). The cDNA library with a size of more than 1×10⁶ clones could ensure every single-copy mRNA contained therein. The size of the cDNA library from adult human aorta as constructed in the invention was 2.4×10⁶ individual recombination clones.

### 1.7 Amplification and Storage of cDNA Library

The constructed cDNA library could be used for direct screening, but it was unstable. Since the non-wild-type bacteriophages tended to lose their activities, it was necessary to amplify the cDNA library for conveniently repeated screening. XL1-Blue MRF′ host cells were transfected with 5×10⁴ bacteriophage and spread on the plate, then incubated for 8-10 hours at 37□. 8 mL of SM buffer was added to 150 mm plate, cultured overnight at 4□, centrifuged and supernatant was collected, and then the amplified cDNA library was obtained. 0.3% chloroform was added to supernatant and stored at 4□. For long term storage, 7% dimethyl sulfoxide (DMSO) was added and frozen at −70□ for storage.

### Example 2

### Cloning of New Full-Length cDNA

### 2.1 Amplification of Random Clones by Polymerase Chain Reaction (PCR)

cDNA library was spread on the plates so that the density of plaque was from 200 to 500 clones in each plate(150 mm). Clear, individual plaque was picked and transferred to a sterile centrifuge tube containing 75 μL of SM buffer and 5 μL of chloroform and placed at 4□. Prior to use, the mixture was mixed and centrifuged, and 5 μL of supernatant was taken out and amplified by PCR using the 3′ primer (5′-CCAAGCTCGAAATTAACCCTCAC-3′) and 5′ primer (5′-CAGTCAATTGTAATACGACTCACT-3′) of ZAP bacteriophage vector in a total volume of 50 μL. Amplification was performed using a program with a pre-denaturing of 3 minutes at 94□, and 30 cycles of 45 seconds at 94□, 30 seconds at 55□, and 3 minutes at 72□ with a final extension at 72□ for 5-10 minutes. The amount of PCR products was estimated according to the brightness of electrophoresis bands in 1% agarose gel.

### 2.2 Sequencing of Expressed Sequence Tag (EST)

ABI PRISM™ 377 DNA sequencer and BigDye™ Terminator Ready Reaction Mix were purchased from Perkin-Elmer Co. of USA. Sequencing was performed by the method of dideoxy-mediated chain termination under the recommended conditions.

Sequencing was performed by using non-radioactive CY5-fluoroscein as a label, and general sequencing primers of T₃ (5′-ATT AAC CCT CAC TAA AGG GA-3′) and T₇ (5′-TAA TAC GAC TCA CTA TAG GG-3′) with the amount of 5 pmol/μL in each sample, and PCR product as template with the amount of 30-100 ng. PCR amplification was performed using a program with a pre-denaturing of 3-5 minutes at 94□, and 20 cycles of 30 seconds at 94□, 15 seconds at 50□, and 1 minute at 72□,l and then 15 cycles of 30 seconds at 94□, and 1 minute at 72□ with a final extension at 72□ for 5 minutes. The DNA product was electrophoresised on a 6% polyacrylamide gel containing 8 mol/L urea for 250-300 minutes with a voltage of 1500V and power of 34W (ALF Express™ DNA Sequencer (Pharmacia Co., Sweden).

### 2.3 Bioinformatics Analysis of EST

EST of each cDNA clone was searched for homology in GenBank/EMBL/DDBJ databank using the BLAST (Basic Local Alignment Search Tool) software of NCBI (http://www.ncbi.nlm.nih.gov). The EST of Fwa116 of the invention was judged as a new EST according to the criteria of BLAST (the general criteria adopted by most of laboratories worldwide), in which the sequence with homologue sequence of less than 100-200 bp and with the score of less than 100 was a new EST.

### 2.4 Sequencing by EST Primer Walking

### 2.4.1 Cyclization of Bacteriophage ZAP in vivo

Bacteriophage ZAP vector could transfer the fragments of interest directly from bacteriophage λ vector to plasmid in the host cell, and create cyclized bacteriophage particle PBR-CMV (carrying the early promoter of CMV). Cyclization was performed with reference to the instructions (ZAP Express™ cDNA Synthesis Kit and ZAP Express™ cDNA Gigapack™ Gold Cloning Kit).

300 μL of E. coli XL1-Blue MRF′ strain (OD₆₀₀=1.0) were co-transfected with 3 μL of bacteriophage stored at 4□ carrying the new EST and 1 μL of helper bacteriophage (a kind of bacteriophage mutant with extremely low self-replication activity, which can provide proteases and coat proteins for replication and package of plasmid DNA in host cells). The single strand DNA was obtained, and then transfected into E. coli XLOLR strain (supplied by kit). 5 mL of LB medium, 25 μL of kanamycin was added to the test tube and the culture was carried out for 12-16 hours at 37□.

### 2.4.2 Extraction and Identification of Plasmid

Plasmid was purified using QIAPrep Spin Miniprep kit (QIAGEN Inc., Germany). Bacterial cells cultured overnight were collected by centrifugation at 2,400 rpm for 10 minutes at 4□. Supernatant was discarded, and cell pellets were suspended in 25 μL of buffer P1 (100 ul/mL RNaseA, 50 mM Tris/HCl, 10 mM EDTA, pH8.0) and transferred to a 1.5 mL steriled centrifuge tube. 250 μL of buffer P2 (200 mM NaOH, 1% SDS) was added and mixed well by inverting the tube several times. 350 μL of buffer P3 (3.0M KAc, pH5.5) was added and mixed by shaking the tube 4-6 times immediately. The mixture was centrifuged at 12,000 rpm for 10 minutes (SORVALL® Mc12V desk centrifuge). The supernatant was collected and transferred to a minipurification column (QIAPrep Spin Miniprep) fitted with a collecting tube at the bottom, then centrifuged at 12,000 rpm for 30-60 seconds. 0.75 μL of buffer TE (10 mM Tris/HCl, 1 mM EDTA, pH8.0) was added to the column and centrifuged at 12,000 rpm for 30-60 seconds. The collecting tube was removed and the QIAPrep Spin Miniprep column was fitted with a steriled centrifuge tube at the bottom. 50 μL of buffer EB (10 mM Tris-HCl, pH8.5) or deionized water was applied to the QIAPrep Spin Miniprep column and held for 1 minute, then centrifuged for 1 minute, and purified DNA was collected.

3 μL of 10× digestion buffer, 1 μL of Not I (15 u/μL), 1 μL of EcoR I (15 u/μL), 1 μL of BSA, 2 μL of DNA and 22 μL of deionized water were added to a centrifuge tube with a total volume of 30 μL, and incubated for 4-6 hours at 37□. After digesting, the size of plasmid was identified.

### 2.4.3 Sequencing of the New Full-Length cDNA

ABI PRISM™ 377 DNA sequencer and BigDye™ Terminator Ready Reaction Mix (Perkin-Elmer Co., USA) were used to the sequencing of positive clones of PBK-VMV.

Reaction mixture contained 4 μL of BD, 2 μL of BOB (400 mM Tris-HCl, pH9.0, 10 mM MgCl₂), 2 μL of primer (5pmol/μL), 30-100 ng of template DNA, and water was added to the final volume of 20 μL. The full-length sequences of cDNA were determined by the method of walking sequencing (Primer Walking), in which the sequencing primer in the next round was determined by the terminal bases of products obtained in the last round. PCR primers were designed by using Oligo 14.0 software.

### Results

As shown in SEQ ID NO:1, full-length Fwa116 cDNA consisted of 2546 base pairs. According to the Kozak Rule, the deduced initial codon was located on the position of nucleotides 1002 to 1004 (ATG), the stop codon on the position of nucleotides 2259 to 2261 (TGA), and the open reading frame was found between nucleotides 1002 and 2261. The results of BLAST analysis showed that this gene was located on chromosome 17.

Homology analysis of protein revealed 70% of homology between the mature polypeptide of Fwa116 and C53 protein, the cdk5 activator binding protein. The deduced Fwa116 protein consisted of 409 amino acids, with the sequence characterized as follows: (A) N- glycosylation site at amino acids 148 to 151 (NYTV); (B) cAMP- and cGMP-dependent protein kinase site at amino acids 405 to 408 (KRYS); (C) myristoylation sites at amino acids 13 to 18 (CVSWAE), 157 to 162 (GTEPSV), 193 to 198 (GTDSGI), 204 to 209 (GIDWGI), and 222 to 227 (GIDWGD); (D) protein kinase C sites at amino acids 41 to 43 (SLK), 57 to 59 (SRK), 77 to 79 (SCK), 339 to 341 (SPR), 404 to 406 (SKR), and 408 to 410 (SGR); (E) casein kinase II sites at amino acids 15 to 18 (SWAE), 57 to 60 (SRKE), 113 to 116 (SLGE), 131 to 134 (SPTE), 150 to 153 (TVYE), 156 to 159 (TGTE), 161 to 164 (SVVE), 235 to 238 (TVLE), 255 to 258 (TLLE), and 316 to 319 (SVLE); (F) leucine zipper motifs at amino acids 270 to 291 (LMELEIF, LAQRAVE, LSEEADVL), and 277 to 298 (LAQRAVE, LSEEADV, LSVSQFQL).

### Example 3

### Distribution of Fwa116 in Normal Tissues

### 3.1 Materials

Multiple tissue northern membrane (MTN membrane) containing mRNAs from 12 tissues was purchased from Clontech Co. (USA) and used to detect the distribution of Fwa116 gene in normal tissues. β-actin, a house-keeping gene which was expressed uniformly in various tissues, was used as control in this experiment.

### 3.2 Northern Blotting

Northern blotting was performed with reference to the methods in “Modem Laboratory Techniques of Molecular Biology” (2nd edition, Lu Shengdong ed., Chinese Xiehe Medical University Press, 1999). The method was described in detail as follows:

### 3.2.1 Extraction of Total RNA

0.5 g of heart tissues of normal adult human was weighed and transferred to a 50 mL centrifuge, then 10 mL of GTC was added and homogenized. An equal volume of water-saturated phenol was added to the homogenate and vertexed vigorously for 15 seconds, and then placed on ice for 20 minutes. The tube was centrifuged at 12,000 rpm for 25 minutes at 4□. The supernatant was transferred to a fresh tube, and an equal volume of isopropanol was added and the mixture was allowed to precipitate for 1 hour at −20□. The tube was centrifuged at 12,000 rpm for 25 minutes at 4□, and then the supernatant was discarded. The precipitate was redissolved in 5 mL of GTC and the above steps were repeated. The final precipitate was washed with 1 mL of pre-cooled 75% ethanol, air-dried, and dissolved in DEPC-treated water. Optical density was measured at 260 nm and 280 nm.

### 3.2.2 Electrophoresis Through Formaldehyde Denatured Gel

0.6 g of agarose was weighed and added to 52.2 mL of DEPC-treated water, and dissolved by heating. When cooled to 65□, 6.0 mL of 10×MOPS and 1.8 mL of formaldehyde was added to produce gel, 4.5 μL (40-60 μg) of total RNA was taken out, to which 2.0 μL of 10×MOPS, 3.5 μL of formaldehyde, and 10 μL of formamide were added, mixed evenly, denatured for 15 minutes at 65□, and chilled on ice for 1 minute. 2.0 μL of loading buffer was added to the sample and mixed. The gel was pre-run for 5 minutes at 50V, and then the sample was loaded. After the dye had been migrated into the gel completely, the voltage was reduced to 40V. Running buffer was mixed in an interval of 10 minutes.

### 3.2.3 Membrane Transferring

The electrophoresis was stopped when the bromophenol blue migrated to the bottom of the gel, and then the gel was photographed. The gel was rinsed with DEPC-treated water for several times. The gel was treated with 50 mM NaOH for 45 minutes, and then with 20×SSC for 45 minutes. The nylon membranes were soaked in the deionized water and then treated with 20×SSC for 45 minutes. A piece of filter paper was placed on the support, and soaked with 20×SSC, then all the air bubbles were removed. The gel was placed on the support in an inverted position, and covered with a plastic film hollowed at the center. The membrane was carefully placed on the top of the gel and all the air bubbles were removed. Two pieces of filter paper were wetted with 20×SSC and placed on top of the membrane, and any air bubbles were removed. A stack of paper towels (10 cm high) were placed on the filter paper and pushed down with a 500 g weight. The membrane transferring was allowed to continue for 16 hours, during which new paper towels were replaced 2-3 times. The membrane was carefully taken away and photographed, then soaked in 6×SSC for 5 minutes. The membrane was cross-linked by UV irradiation and heated at 80□ for 1 hour, then stored at 4□ until use.

### 3.2.4 Preparation of Template for Hybridization

Upstream primer: 5′-C GTCGAC GCTCTTCACCACCACAAAGGATG-3′, where the italics was protection base, the bold was a Sal I site, the underline is complementary to the nucleotide sequence of Fwa116 at position of 982 to 1001; Downstream primer: 5′-AA GCGGCCGC TGTCACAGAGAGGTTCCCATCA-3′, where the italics was protection base, the bold was a Not I site, the underline was complementary to the nucleotide sequence of Fwa116 at position of 2242 to 2263.

PCR reaction mixture contained 5.0 μL of 10×buffer, 2.0 μL of DNA, 3.0 μL of each of upstream and downstream primer, 1.0 μL of Taq polymerase, 2.0 μL of Fwa116 sequencing plasmid (template), and 34 μL of sterile water. PCR amplification was performed using a program with a pre-denaturing of 3 minutes at 94□, and 30 cycles of 20 seconds at 94□, 30 seconds at 60□, and 80 seconds at 72□, with a final extension at 72□ for 7 minutes. The PCR product was purified using ammonium acetate/ethanol (1:5), and dissolved in 50 μL of TE. The amount of PCR product was quantitatively determined by electrophoresis on agarose gel, and the product was diluted to a concentration of 25 ng/μL.

### 3.2.5 Hybridization

Labeling of the probe: 25 ng of PCR product (denatured for 4 minutes by heating at 98□, and chilled for 2 minutes on ice), 10 μL of 5×labeling buffer, 2.0 μL of dNTP (without dCTP), 2.0 μL of BSA, 1.0 μL of Klenow enzyme, and 5.0 μL of [α-³²P]dCTP were added to a 0.5 mL centrifuge tube, and the total volume of mixture was adjusted to 50 μL with RNase-free water. Reaction was performed for 1-3 hours at room temperature.

Prehybridization: The membrane was soaked with 6×SSC for 5 minutes, then the membrane was placed against the wall of the hybridization tube, and any air bubbles were removed. 6 mL of hybridization buffer (purchased from Clontech) was added to the tube and incubated for 1 hour at 68□.

Hybridization: Poured out hybridization buffer, and 6 mL of preheated hybridization buffer was added to the tube. Probe (denatured for 4 minutes by heating at 98□, and chilled for 2 minutes on ice) was added and hybridization was carried out for 3 hours at 68□.

Washing membrane: At first, the membrane was washed for 10 minutes at room temperature with 200 mL of washing solution I (2×SSC, 0.05% SDS), repeated for 4 times, and then with 200 mL of washing solution II (0.1×SSC, 0.1% SDS) for 20 minutes at 50□ and for 20 minutes at 56□.

Pressing film: Solution on the membrane was sucked dry with filter paper. The membrane was wrapped with a piece of preservative film, and stuck on a piece of filter paper with the same size as X-ray film, then the film was pressed. The membrane was exposed to X-rays for a proper time and the film was processed.

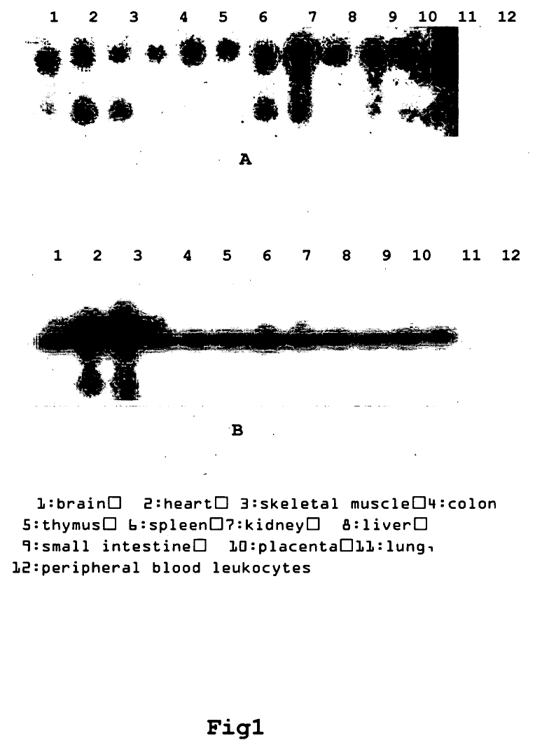

Results: As shown in FIG. 1, Fwa116 gene was widely distributed among various tissues. Upper bands were represented for the transcript of 3.4 Kb, and lower bands for the transcript of 1.8 Kb. The transcript of 3.4 Kb was highly expressed in liver and peripheral blood white blood cells, and the transcript of 1.8 Kb was relatively highly expressed in the heart, skeletal muscle, kidney, and liver.

### Example 4

### Distribution of Fwa116 Gene in Different Tumor Cell Lines

### 4.1 Materials

Hybridization membrane containing mRNAs from 8 tumor cell lines was purchased from Clontech Co. (USA) and used to detect the distribution of Fwa116 gene in different tumor cell lines. β-actin was used as control in this experiment.

### 4.2 Northern Blotting

### See Example 3.2.

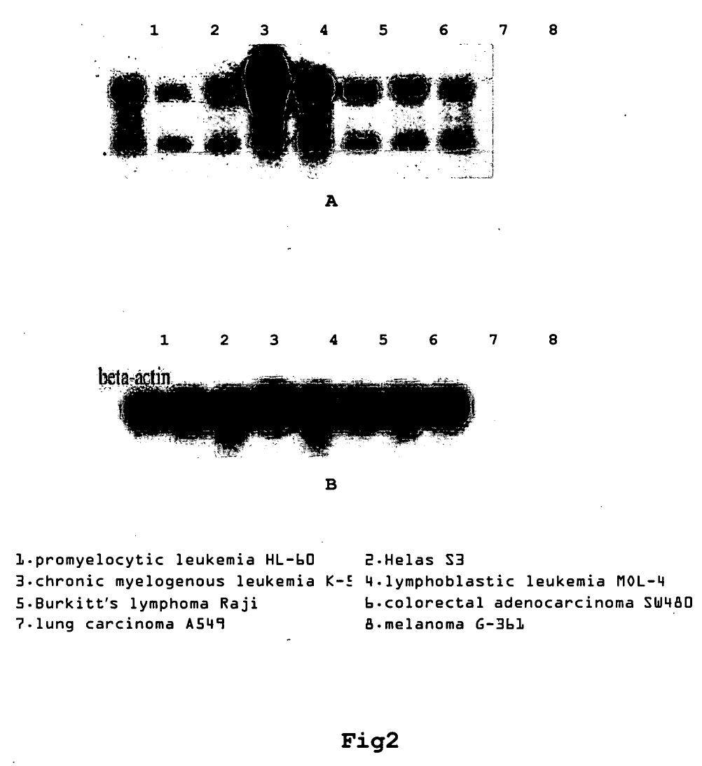

Results: As shown in FIG. 2, β-actin was expressed uniformly in several tumor tissues (FIG. 2B); Fwa116 gene could be expressed in all 8 tumor cell lines tested, in which the highest expressions were observed in lymphoblast leucocytoma cell line, Burkitt's lymphomata Raji cell line, and promyelocytic leukemia cell line.

### Example 5

### Effects of Oxidized-Low Density Lipoprotein (ox-LDL) on the Expression of Fwa116 in Human Blood Vessel Endothelia Cell

Oxidized-low density lipoprotein (ox-LDL) is a molecular risk factor leading to atherosclerosis, which can induce the expressions of various genes and lead to damages and inflammatory reactions of blood vessel endothelia cell. This experiment was intended to investigate the relations between ox-LDL and the expression of Fwa116 in human blood vessel endothelia cells.

### 5.1 Preparation of Oxidized-Low Density Lipoproteins (ox-LDL)

Low density lipoproteins (LDL) with a density of 1.019 to 1.063 obtained by density gradient centrifugation were oxidized with Cu²⁺ for 24 hours, filtrated through a 0.22 μm filter membrane, and stored at 4□ in a dark place. The value of TBARS was determined and the ox-LDL was separated by electrophoresis on agarose gel. More than 10 nmol/mg of TBARS value and the increase of electrophoretic mobility indicated successful oxidation.

### 5.2 Culture of Human Endothelia Cell

The frozen tube containing human blood vessel endothelia cells was taken out from liquid-nitrogen, and put rapidly into a water bath at 37□]. After being melted completely, the cell suspension was inoculated to a 25 mL culture bottle containing 5 mL of RPMI or DMEM with 10% FES. Culture was carried out overnight in an incubator at 37□ and 5% of CO₂ concentration. The medium was replaced on the next day.

### 5.3 Subculture

When Cells were grown to substantial confluency (with a covering percentage of around 80%), the original medium was discarded. After Cells were rinsed twice with 1× PBS (pH7.4), 0.125% trypsin was added, and the cells were digested for 5-10 minutes at 37□. When the cells were contracted and became round and partly floated while observed under a microscope, digestion was stopped by adding a little medium containing 10% FBS. The cells were washed by blowing the cell surface with a pipette repeatedly, and the digesting solution was collected and centrifuged at 1,000 rpm for 30 seconds. Supernatant was discarded and the cells were washed by adding medium containing 10% FBS. After being mixed evenly, 0.1 mL of cell suspension was taken out and diluted with 1×PBS to 1.0 mL. The number of cells was counted, and the cell suspension was inoculated to a culture bottle containing 5 mL of RPMI or DMEM with 10% FES to a concentration of 100,000 cells/mL. Culture was carried out in an incubator at 37□ and 5% of CO₂ concentration. By this method cells were subcultured for 3 generations until the desired number was achieved. When Cells were grown to substantial confluency (with a covering percentage of around 80%), the original medium was discarded. The medium containing 0.4% FBS was added and culture was continued for another 24-72 hours until the cells were in still growth phase.

### 5.4 Stimulating Cell with ox-LDL and Soybean Genistein

Cells were stimulated with 200 μg/mL of ox-LDL for 12, 18 and 36 hours, respectively. The soybean genistein was added simultaneously to a final concentration of 100 μM at 12 hours. Culture was carried out in an incubator at 37□ and 5% of CO₂ concentration. Cells were collected by GTC, and the supernatant was reserved. Three bottles of cells were collected for Northern blotting.

### 5.5 Northern Blotting

Three bottles of cells were collected from the last step. For other steps see example 3.2.

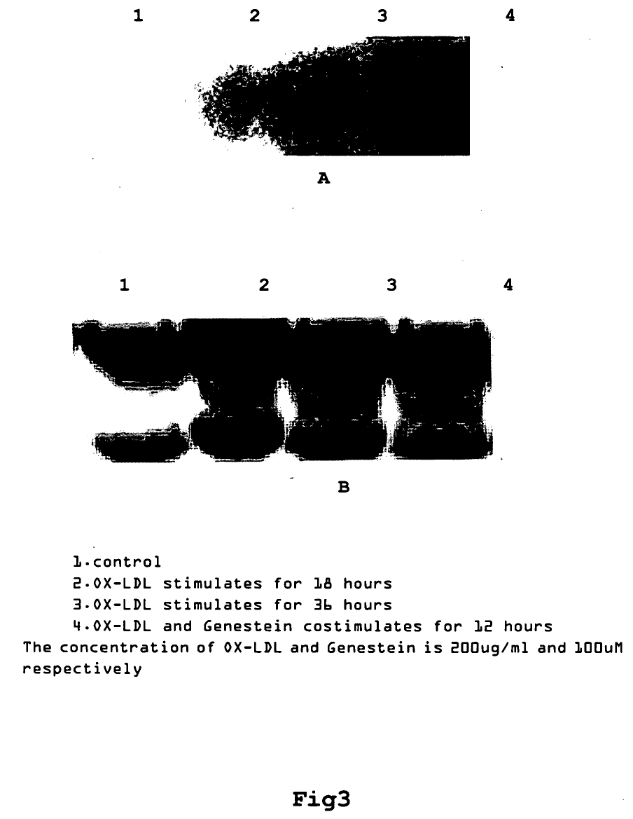

Results: As shown in FIG. 3, ox-LDL can stimulate the expression of Fwa116 in endothelia cells. When the cells were treated with ox-LDL and soybean genistein simultaneously, the expression of Fwa116 increased by more than 10 fold.

The results above suggested that the expression of Fwa116 induced by ox-LDL was a compensation reaction to the antioxidation aroused by ox-LDL and Fwa116 was possibly an antioxidization factor. The protective effects against arteriosclerosis caused by the antioxidation of soybean genistein, were achieved probably by up-regulating the Fwa116 gene. Therefore, up-regulating the Fwa116 product was able to protect blood vessels against certain risk factors of arteriosclerosis.

### Example 6

### Expressions of Fwa116 Gene in the Hearts of Normal, Chronic Heart Failure and Sub-Acute Heart Failure Animals

### 6.1 Establishment of the Model of Chronic Heart Failure Rat

Fifty male Sprague-Dawley (SD) rats (each 250-300 g) were purchased from the animal feeding station of our Academy. Standard feeds were provided by the Beijing Center of animal and drinking water adopted tap water. Animals ingested water and feeds voluntarily.

The rats were weighed out, and ketamine and diazepam were injected intraperitoneally to the rats causing general anesthesia based on the weight of body, then intubated in trachea and connected to a miniature animal breathing machine. Under the monitor of electrocardiograph, the bosom at left was opened; the descending anterior branch of left coronary artery with stylolite of 6-10 size was deligated; the bosom was closed. Evident ascension of ST phase on electrocardiogram indicated successful deligation. Breathing machine was removed when rats awoke. The feeding conditions after operation was the same as before the operation. Model was established 50 days later.

### 6.2 Establishment of the Model of Sub-Acute Heart Failure Rat

Ten male Wistar rats (each 250 g) were purchased from No.301 hospital of People's Liberation Army. The feeding conditions were the same as that for the model of chronic heart failure rat.

Noradrenaline was injected intraperitoneally with a dose of 30 mg per day per rat. Injection was carried out for four successive days, and the model can be used on the fifth day.

### 6.3 In situ Hybridization of RNA-RNA

The technology of in situ hybridization is based on the principle of base complementarity, where the genes and expressed products in cells are visualized by specifically hybridizing of probes to specific mRNA or DNA in cells. The method provides high sensitivity, and can detect the genes only expressed instantaneously during the course of tissue differentiation. DIG RNA Labeling kit (Cat. No. 1175025) was purchased from Boehringer Mannheim Co. (Germany).

### 6.3.1 Preparation of Probe

An Fwa116 nucleic acid fragment of 200-300 bp in length (the nucleotides 1371-1632) was cloned into T vector by PCR amplification with specific primers. The recombinant plasmids were amplified and purified, and then PCR amplification was carried out with T7 and SP6 primers. The direction of gene inserted was determined by PCR amplification with specific primer and vector T7 and SP6 primer. The sequences of mRNA and anti-mRNA were determined. Antisense probe was synthesized by T7 RNA polymerase, and sense probe by SP6 RNA polymerase to check the reliability of hybridization system.

### 6.3.2 Labeling of Probes

The transcribed linear DNA was extracted with phenol/chloroform, and precipitated by ethanol. A RNase-free centrifuge tube was placed on ice, to which 1 μL of the purified linear DNA, 2 μL of labeled NTP mixture, 2 μL of loxtranscription buffer, 1 μL of RNase inhibitor, 2 μL (40 U) of SP6 or T7 RNA polymerase were added with a total volume of 20 μL, and then mixed evenly and centrifuged. 20 u of RNase-free DNase I was added, and was incubated for 15 minutes at 37□. Polymerization was stopped by adding 2 μL of 0.2 mol/L EDTA (pH8.0).

To the above mixture, 2.5 μL of 4mol/L LiCl and 75 μL of ethanol (−20□) were added and mixed well, and then placed for at least 30 minutes at −70□ (or at least 2 hours at −20□). Centrifuged at 12,000 rpm, washed with 70% cold ethanol, and dried. 100 μL of DEPC-treated water and 20 u Rnase were added, the mixture was placed for 30 minutes at 37□, fractionally packed and stored at −20□.

### 6.3.3 Treatment of Glass Slide and Preparation of Sample

The glass slides were completely rinsed and sterilized for 30 minutes under high pressure. The glass slides were soaked in solution containing 0.01% polylysine, dried, and stored at 4□ until use.

Fresh samples were from the aorta and heart blood vessels of rats (from normal, chronic heart failure and sub-acute heart failure model animals) with a size of 0.4 cm×0.4 cm. The samples were washed twice with 1×PBS, then placed on the fixed head of freezing microtome. The slices at thickness of 8-10 μm were placed on the glass slides containing 1 mg/mL polylysine and dried. Samples were fixed for 15-20 minutes with 4% paraformaldehyde, and then washed twice with 1×PBS for 5 minutes once.

### 6.3.4 Treatment Before Hybridization

Samples were soaked in 0.2N HCl for 25 minutes, and in 0.3% Triton X-100 for 5 minutes, and then washed twice with 1× PBS for 5 minutes once. After being fixed with 4% paraformaldehyde for 6 minutes, and then washed twice with 1×PBS for 5 minutes once. 5 mL of acetic anhydride was added to 1000 mL of 0.1 mol/L triethanolamine (pH8.0) solution. After they were dissolved completely, the slices was soaked for 10 minutes. All reactions described above were carried at room temperature.

### 6.3.5 Hybridization

Hybridizing solution contained 5 mL of deionized formamide, 2.5 mL of 20×SSC, 500 μL of 100×Denhardt's solution (10 g saccharosan, 10 g polyvinyl pyrrolidone, 10 g BSA, 500 mL of sterilized double-distilled water), 500 μL of 10% SDS, 100 μL of 10 mg/mL denatured herring sperm DNA, and 400 μL of DEPC-treated water.

Slices were taken out from the acetylated solution, and the liquid around samples was sucked up with filter paper. A little circle around the sample was drawn and 30 μL of pre-hybridization solution was added to the circle, incubated in a wet box for 2 hours at 42□. The pre-hybridization solution was removed and 25 mL of hybridization solution was added to cover the Parafilm, incubated in the wet box for 16 hours at 42□ and the wet box was sealed.

### 6.3.6 Treatment After Hybridization

The glass slides were taken out from the wet box and removed the Parafilm. The glass slides were washed with 2×SSC at room temperature for three times with 10 minutes once, and with 1×SSC at room temperature for three times with 10 minutes once, and with 0.1×SSC at 50□ twice with 15 minutes once.

If the background was too high, the glass slides were put into the solution (0.5 mol/L NaCl, 10 mmol/L Tris-HCl, pH8.0) containing 20 μg/mL RNase, and digested for 30 minutes at 37□. Then the samples were washed with the RNase-free solution for 30 minutes at 37□.

### 6.3.7 Colorimetric Reaction

Glass slides were rinsed in washing solution (1M maleic acid, 0.15M NaCl, 0.3%(V/V) Tween-20). Then they were incubated for 30 minutes in 100 mL of blocking solution (10%(W/V) blocking agent of kit in maleic acid buffer (0.1 mol/L maleic acid, 0.15 mol/L NaCl), diluted 10 times when used). 20 mL of antibody solution was added and incubated for 30 minutes, and washed twice with 100 mL of washing solution for 15 minutes once. 20 mL of detection solution (0.1M Tris-HCl, 0.1M NaCl, pH9.5) was added and remained for 2-5 minutes until equilibrium. Then the mixture was incubated in 10 mL of fresh staining solution (adding 200 μL of NBT/BCIP to 10 mL of detection solution), kept in a dark place without shaking and washed with sterilized double-distilled water or TE.

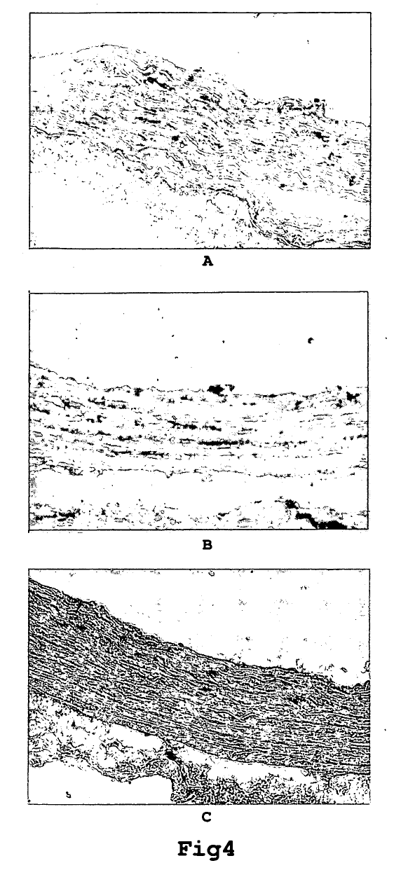

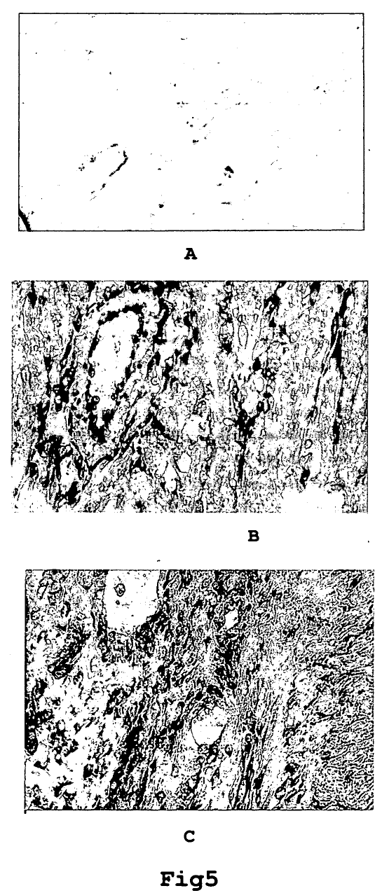

Results: As shown in FIGS. 4 and 5, Fwa116 gene could be expressed in endothelia cell in heart blood vessel and aorta of normal rat, but with a low level (FIG. 4A. FIG. 5A); High level expression of Fwa116 gene in endothelia cell in heart blood vessel and aorta were achieved in the model of sub-acute heart failure rat (FIG. 4B, FIG. 5B) and in the model of chronic heart failure rat caused by deligating coronary artery leading to ventricular aneurysm, Fwa116 gene was expressed highly in aorta and heart blood vessel (FIG. 4C, FIG. 5C).

### Example 7

### Immunohistochemistry Assay

### 7.1 Materials

Tissues of ventricular aneurysm created with human acute myocardial infarction.

### 7.2 Immunohistochemistry Assay

The prokaryotic expression plasmid with correct recombination was used to transfect E. coli BL21 competent cell. The cells were disrupted by ultrasonication. Electrophoresised on 10% denatured polyacrylamide gel and the bands of gene of interest were identified. Fusion protein was obtained from inclusion bodies by the methods of sonication and electrical elution. The protein of interest was identified by Western blotting and used to immunize animal. The serum was obtained and used as the first antibody in this experiment.

Samples embedded in paraffin were sliced with a thickness of 5 μm, and slices were adhered on APES glass slides coated with polylysine. The slides were baked for 2 hours at 75□, dewaxed with dimethyl benzene, treated with gradient ethanol, and sliced into water. The slices were placed in 3% H₂O₂ for 10 minutes, rinsed with distilled water for three times, soaked in EDTA antigen restoring solution (pH8.0), and then baked for 10 minutes in microwave oven (96□-98□). They were cooled at room temperature for 20-30 minutes, rinsed with distilled water for three times, soaked in 1×PBS buffer for 5 minutes, and then soaked in 10% normal rabbit serum for 20 minutes. Added first antibody diluted properly, incubated overnight at 4□. Rinsed with 1×PBS for three times, added biotin-labeling goat-anti-rabbit second antibody, placed for 10 minutes at room temperature. Rinsed with 1×PBS for three times, added enzyme-linked streptavidin third antibody, placed for 10 minutes at room temperature. Rinsed with 1×PBS for three times, stained with DBA. Rinsed with distilled water for three times, stained the nucleus with hematoxyllin, dehydrated, and mounted the slices.

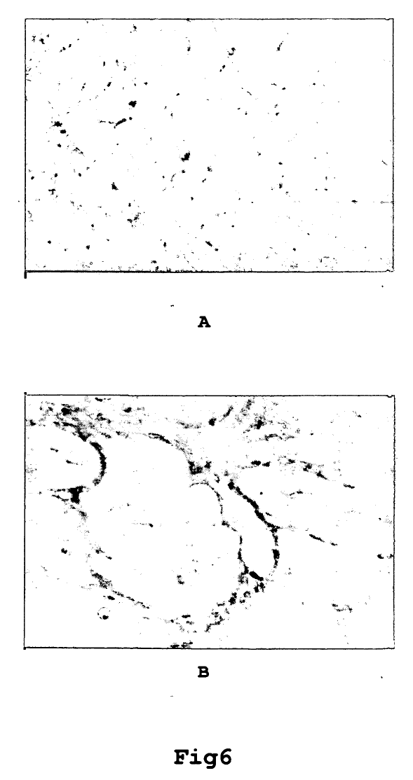

Results: As shown in FIG. 6, Fwa116 gene was expressed highly in endothelia cell of blood vessel from tissues of ventricular aneurysm created with human acute myocardial infarction.

### Example 8

### Inducing the Proliferation of ECV304 Cell by Over-Expression of Fwa116 Gene

### 8.1 Materials

ECV304 cell line was purchased from ATCC.

### 8.2 Culture of Cell and Transfection of Fwa116 Gene

### 8.2.1 Culture of ECV304 Cell

Cells were cultured in 6-well culture plates with 5×10⁵ cells in each well, in 0.5 mL of RPMI1640 medium (GIBCO) containing 10% fetal calf serum.

### 8.2.2 Transfection with Fwa116 Gene

The open reading frame (ORF) of Fwa116 was constructed in pcDNA3.1/Myc-His(−)A vector (CLONTECH). After cells had grown for 20 hours, the vector carring Fwa116 gene was introduced in ECV304 cells using the method instructed by Clonfection kit (CLONTECH, USA). 48 hours after transfection, the clones that could express Fwa116 gene steadily were screened with G418 (200 μg/mL). The cells transfected with empty vector were used as control. The clone with the highest expression of Fwa116 gene was screened by Northern blotting, and reproduced to observe the growth curve of the cell. The clone with steady and high expression of Fwa116 gene, was inoculated to 24-well culture plate, and cultured in RPM11640 medium containing 10% FBS. Medium was replaced in every 48 hours. Cells were taken out from 3 wells per group in every 24 hours, digested with protease, and then counted.

Method for digestion with protease: Removed the medium, washed the cells twice with 1×PBS (0.5 mL/cm² of culture plate), added 1×PBS containing 0.125% trypsin to digest the cells for 5 minutes, blew the cells repeatedly by pipette and mixed evenly. Then the number of cells was counted on a blood cell counting plate.

Method for counting cells: Cells were dropwised in the blood cell counting plate, put under the optical microscope (Nikon, Japan) and the number of cells was counted. The number of cells was calculated according to the formula as follows:

The number of cells/mL=Number of cells in four large lattices/4×10,000×dilution factor

### Results

Results of counting of the cells (×10⁴)

| | ECV304 | ECV304 | ECV304 |
|---|---|---|---|
| | transfected with | transfected with | transfected with |
| | empty vector | Fwa116 gene | Fwa116 gene |
| 1^{st} day | 1.0625 | 2.438 | 2.250 |
| 2^{nd} day | 5.125 | 6.687 | 6.875 |
| 3^{rd} day | 7.688 | 16.000 | 14.563 |
| 4^{th} day | 18.500 | 43.5975 | 39.8475 |
| 5^{th} day | 33.500 | 60.500 | 65.500 |
| 6^{th} day | 49.000 | 105.500 | 102.875 |

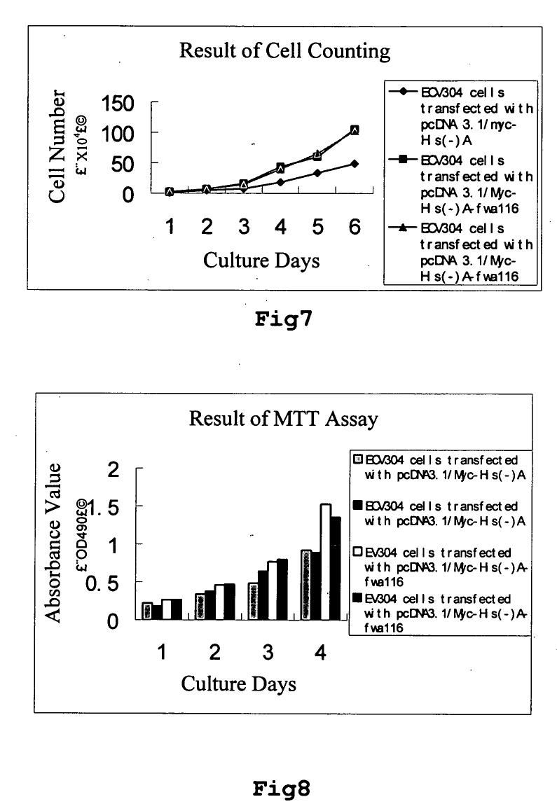

As shown in FIG. 7, beginning on the 4^{th} day, the proliferation of cells highly expressing Fwa116 gene was two times higher than that of control cells transfected with empty vector (P<0.05).

### Example 9

### Surviving Test (MTT-Test) of Cell

ECV304 cells were cultured in 96-well plates with an inoculating concentration of 5000 cells per well. Cells were picked up at 24 hour, 48 hour, 72 hour and 96 hour after inoculation, respectively, and stained with 20 μL of MTT (5 ng/mL, Sigma), then observed at 490 nm using a Bio-Rad Microplate Reader (USA), the stained cells were surviving cells.

### Results

Experimental data from MTF-test was shown in the following table:

| | Transfected | Transfected | Transfected | Transfected |
|---|---|---|---|---|
| | with empty | with empty | with Fwa116 | with Fwa116 |
| | vector 1 | vector 2 | gene 1 | gene 2 |
| 24 hours | 0.220 | 0.183 | 0.266 | 0.266 |
| 48 hours | 0.341 | 0.378 | 0.462 | 0.473 |
| 72 hours | 0.488 | 0.645 | 0.769 | 0.800 |
| 96 hours | 0.919 | 0.888 | 1.527 | 1.354 |

Mean ± SD, n = 3
*Compared to the control (clones transfected with empty vector): P < 0.05
As shown in FIG. 8, the proliferation of cells highly expressing Fwa116 gene was significantly faster than that of control cells transfected with empty vector (P < 0.05).

The foregoing examples are intended merely to illustrate a few aspects of this invention but not to limit the scope of the invention. Methods and materials functionally similar to those of the invention are included in the scope of the invention. In fact, it is evident that to one skilled in the art, numerous changes and modifications may be made to the embodiments of the invention based on the description and drawing in the context. It is intended that all such variations fall within the scope of claims of the invention.

What is claimed is:

1. An isolated polynucleotide comprising one member selected from the group consisting of:
(a) a polynucleotide, encoding the polypeptide of SEQ ID NO:2;
(b) a polynucleotide, being a naturally occurring variant of polynucleotide (a);
(c) a polynucleotide, hybridizing with (a) and having at least 85% of homology with (a).

2. The polynucleotide of claim 1, wherein said polynucleotide is DNA.

3. The polynucleotide of claim 1, wherein said polynucleotide is RNA.

4. The polynucleotide of claim 1, wherein said polynucleotide is a genomic DNA.

5. The polynucleotide of claim 1, wherein said polynucleotide has a sequence of SEQ ID NO:1.

6. The polynucleotide of claim 1, wherein said polynucleotide comprises nucleotides 1002-2261 of SEQ ID NO:1.

7. The polynucleotide of claim 2, encoding the polypeptide of SEQ ID NO:2.

8. A vector comprising the DNA of claim 2.

9. A host cell transformed or transfected with the vector of claim 8.

10. A method for preparing a polypeptide, comprising the polypeptide encoded by said DNA as expressed in the host cell of claim 9.

11. A polypeptide comprising one member selected by the group consisting of:
(a) a polypeptide, having the deduced amino acid sequence of SEQ ID NO:2;
(b) a polypeptide, being an active fragment, analogue or derivative of (a);
(c) a polypeptide, having at least 85% of homology with the amino acid sequence of SEQ ID NO:2.

12. An antibody against the polypeptide of claim 11.

13. A compound inhibiting the activation of the polypeptide of claim 11.

14. A pharmaceutical composition comprising an effective amount of the polypeptide of claim 11 or the active fragment thereof and one or more pharmacologically acceptable carriers or excipients.

15. Use of the polypeptide of claim 11 for preparing the pharmaceutical composition in the treatment of cardiovascular inflammatory arteroscierosis and tumors.

16. The use of claim 15, wherein said cardiovascular inflammatory arterosclerosis includes cerebral apoplexy, coronary heart disease, angina pectoris and myocardial infarction.

17. A method for treatment of a patient in need of Fwa116 comprising administration of a therapeutically effective amount of the-polypeptide of claim 11 to the patient.

18. The method of claim 17 comprising administration of the DNA encoding said polypeptide to the patient and expression of said polypeptide in the body of the patient.

19. A method for diagnosis of a disease or susceptibility of the disease comprising determination of the mutation of the polynucleotide of claim 1.

20. A method for diagnosis comprising analysis of whether the polypeptide of claim 11 is present in the sample from the host cell or not.
